(19) United States
(12) Patent Application Publication
(10) Pub. No.: US 2025/0282701 A1
(43) Pub. Date: Sep. 11, 2025

(54) A PROCESS FOR THE DIRECT CONVERSION OF SUGAR TO VALUE ADDED PRODUCTS

(71) Applicant: COUNCIL OF SCIENTIFIC AND INDUSTRIAL RESEARCH, New Delhi (IN)

(72) Inventors: Banu MARIMUTHU, Pune (IN); Sreejith SREEKANTAN, Pune (IN); Arun Arunima Balachandran KIRALI, Pune (IN)

(73) Assignee: COUNCIL OF SCIENTIFIC AND INDUSTRIAL RESEARCH, New Delhi (IN)

(21) Appl. No.: 18/859,029

(22) PCT Filed: Apr. 21, 2023
(86) PCT No.: PCT/IN2023/050390

§ 371 Date: Oct. 22, 2024

(30) Foreign Application Priority Data
Apr. 22, 2022 (IN) 202211024076

(51) Int. Cl. C07C 29/132; B01J 21/04; B01J 23/883; B01J 35/64; C07C 27/04; C07C 51/31

(52) U.S. Cl. CPC C07C 29/132; B01J 21/04; B01J 23/883; B01J 35/64; C07C 27/04; C07C 51/31

(57) ABSTRACT

An efficient one step, one pot process of synthesis of value-added products such as diols, monools and/or acids, preferably 1,2-PG from sugars/polysachhrides is disclosed. The process is catalyzed by a bimetallic catalyst supported on alumina and the catalyst is recyclable. The process of synthesis of the catalyst is a simple process and it converts a high % of the substrates to the desired polyols under mild conditions.

### FIELD OF THE INVENTION

The present invention relates to a process for the direct conversion of sugar into value added products such as 1,2-propanediol, monool and/or acid. More particularly, the present invention relates to one step and one pot process for the direct conversion of sugar into value added products using catalysts based on metal(s) supported on a support.

### BACKGROUND AND PRIOR ART OF THE INVENTION

To provide the higher standards of living and to compete economically with others, both developed and developing nations are consuming non-renewable resources. An improved energy technology with reduced impact on the environment may aid us in coping with the ever increasing energy demand. However, providing much efficient, cleaner, and a renewable energy could mitigate the energy crises and reduce environmental impacts as by being sustainable and lessening green house gas (GHGs) emissions.

Recently, the inventors has reported the selective production of ethylene glycol (EG), one of the most demanding energy chemicals from cellulose and glucose, refer, Sreejith et al. New J. Chem., 2021, 45, 19244-19254 and Arun Arunima Kirali et al. New J. Chem., 2020, 44, 15958-15965. However, the synthesis of the support as per this article requires glucose, templates like CTAB, silica source like TEOS, sodium silicates, acids to maintain pH and NaOH to neutralize and desilication, increasing overall complexity, cost, and number of chemicals which makes the process highly tedious.

Just like EG, 1,2-propane diol (1,2-PDO) is a glycol with a wide range of applications. Though used as an additive in cosmetics, nutritional products, medicines and dyes, it is increasingly used in anti-freeze and de-icing fluids, as well as a component of unsaturated polyester resins, liquid non-ionic detergents, and coolants.

Very little literature exists regarding the direct conversion of sucrose to 1,2-propanediol (1,2-PG). So far the literature known methods reported for the synthesis of 1,2-propanediols is from cellulose, glucose, sorbitol or glycerols. The production of 1,2-PG from glucose and cellulose suffers from the drawbacks such as requirement of high reaction temperatures and pressures, low conversions, comparatively high yield of ethylene glycol, and also the inefficiency of batch reactor to yield high concentration of desired products owing to charring and other side reactions. Therefore, it is highly desirable to develop an economically viable and efficient catalytic process to replace the short comings of the known processes to synthesize 1,2-PG.

Further, the catalysts known in the art for the synthesis of 1,2-PG and/or other value added products involve tedious synthetic processes and are unstable in nature, making them commercially unattractive. Thus, there is a need in the art to provide a catalystic system which will give high conversion of sugar/polysaccharide to the desired value added products at moderate reaction conditions and also possesses good reusability.

India though being an agricultural country and one of the largest suppliers of sucrose from sugarcane, there has been no efforts to commercialize the conversion of sugar to biofuel. So, a process for the efficient conversion of sugar to 1,2-PG as well as other value added products is proposed by the inventors in the present specification.

### OBJECTIVES OF THE PRESENT INVENTION

The main objective of the present invention is to provide a process for the conversion of sugar into value added products such as diols, monools and/or acids by using catalyst containing a non-noble metal(s) supported onto a support.

An another objective of the present invention is to provide a process for the conversion of sucrose into 1,2-propanediol by using catalyst containing a non-noble metal(s) supported onto a support.

Yet another objective of the present invention is to provide a process for the conversion of sugar into value added products with enhanced yield ratio of EG:PG by using catalyst containing a non-noble metal(s) supported onto a support.

### SUMMARY OF THE INVENTION

Accordingly, the present invention provides a process for the direct conversion of substrates such as sugars or polysaccharides into valuable products, said valuable products are glycols, using a catalyst containing non-noble metal(s) supported onto a support to obtain said valuable products with 100% conversion of the substrate.

In an aspect, the present invention relates to a one pot-one step process for preparation of value added products from sugar(s), the process comprising:
mixing a reaction mixture comprising said sugar(s) or polysaccharide(s) as a substrate, and a catalyst containing non-noble metal(s) supported onto a support, in a solvent under H₂ gas, at pressure in the range of 20 to 70 bar, and at a temperature of 20 to 30° C., followed by heating at a temperature in the range of 150 to 220° C. and stirring at a speed in the range of 500 to 1000 rpm for time period in the range of 1 minute to 6.5 hrs to obtain the value added products.

In another aspect, the value added product is selected from polyol/diol, monool (monohydroxy alcohol), acid or mixtures thereof.

In another aspect, the value added products is/are selected from 1,2-propanediol, 1,3-propanediol, ethylene glycol, 1,2-butanediol, 2-propanol, 1-propanol, lactic acid or mixtures thereof.

In another aspect, the non-noble metal(s) loaded onto the support in said catalyst is selected from nickel (Ni), molybdenum (Mo), cobalt (Co), copper (Cu), iron (Fe), tungsten (W), tin (Sn), chromium (Cr), cerium (Ce), alone or combinations thereof.

In another aspect, the support is selected from, but not limited to alumina (γ-Al₂O₃), zeolites and mesoporous carbon.

In another aspect, the process comprises reacting 1-10 wt % of sugar with said catalyst in a ratio of 1:0.2 to 1:1, wherein the catalyst is a bimetallic (M1M2) supported onto a support at a temperature in the range of 150° C. to 220° C., rpm in the range of 700 to 1000 rpm, and pressure in the range of 20-70 bars of hydrogen, to obtain said value added products.

In another aspect, the present invention provides a cost-effective, environmental friendly, stable and reusable catalyst for the efficient conversion of sugar(s) into value added products, wherein said catalyst is M1-M2/Al₂O₃ with the ratios of M1 varying from 3-12%, and M2 varying from 5-30%.

In another aspect, the catalyst is found reusable for consecutive runs without any change in structural properties of metals and/or support, and the leaching of metals.

### BRIEF DESCRIPTION OF THE DRAWING

FIG. 1 shows selectivity of value added products formation using different γ-Al₂O₃ supported Ni—Mo metal catalysts on 2 wt % sucrose at 220° C., 40 bar of H₂ and 1000 rpm for 4.5 hrs.

FIG. 2 shows HPLC graph confirming production of different value added products.

### ABBREVIATIONS USED

1,2-PDO: 1,2-propanediol
1,3-PDO: 1,3-propanediol
EG: Ethyleneglycol
1,2-BDO: 1,2-butanediol
2-PrOH: 2-propanol
PDO: Propanediol
BDO: Butanediol

### DETAILED DESCRIPTION OF THE INVENTION

In an embodiment, the present invention relates to a one pot-one step process for preparation of value added products or diol(s) from sugar(s) or polysaccharide(s), the process comprising: mixing a reaction mixture comprising said sugar(s) or polysaccharide(s) and a catalyst containing non-noble metal(s) supported onto a support, in a solvent under H₂ gas, at pressure in the range of 20 to 70 bar at temperature of 20 to 30° C., followed by heating at a temperature in the range of 150 to 220° C. and stirring at a speed in the range of 500 to 1000 rpm for a time period in the range of 2.5 to 6.5 hrs to obtain said value added products or diol(s).

In another embodiment, the pressure is in range of 30 to 50 bar; temperature for heating is in range of 160 to 180° C.; and stirring speed is in range of 700 to 1000 rpm.

In another embodiment, the value added product is selected from polyol/diol, monool (monohydroxy alcohol), acid or mixtures thereof.

In another aspect, the value added products is/are selected from 1,2-propanediol, 1,3-propanediol, ethylene glycol, 1,2-butanediol, 2-propanol, 1-propanol, lactic acid or mixtures thereof.

In another embodiment, the conversion rate of said sugar(s) into said value added products is 100%.

In another embodiment, the non-noble metal(s) loaded onto the support in said catalyst is selected from nickel (Ni), molybdenum (Mo), cobalt (Co), copper (Cu), iron (Fe), tungsten (W), tin (Sn), chromium (Cr), cerium (Ce), Lanthanum (La) alone or combinations thereof.

In another embodiment, the catalyst used herein is a bimetallic system supported onto a support.

Specifically, the catalyst comprises M1M2 metals supported onto a support.

The M1 is selected from nickel (Ni), cobalt (Co), copper (Cu), and Iron (Fe). The M2 is selected from molybdenum (Mo), iron (Fe), tungsten (W), Lanthanum (La), tin (Sn), Cerium (Ce), and chromium (Cr).

In another embodiment, the weight % of M1 in said catalyst is in range of 3-12 wt. %; the weight % of M2 in said catalyst is in range of 5-30 wt. %; and the weight % of support in said catalyst is in range of 58 to 92 wt. %.

In another embodiment, the support is selected from, but not limited to alumina (γ-Al₂O₃), zeolites and mesoporous carbon.

In another embodiment, the solvent to dissolve the said sugar(s) with a catalyst is selected from but not limited to polar protic solvent. The polar protic solvent is selected from water, methanol and ethanol. In a preferred embodiment, the solvent is water.

In another embodiment, the ratio of said sugar(s) and said catalyst is in range of 1:0.2 to 1:1.

In a preferred embodiment, the sugar is selected from monosaccharides or disaccharides. In another preferred embodiment, the sugar is selected from pentose(s) or hexose(s).

In another embodiment, the polysaccharide is used in the process instead of said sugar as substrate. The polysaccharide is cellulose or starch.

In another embodiment, the process produces diols or monools are selected from 1,2-propanediol, ethylene glycol, n-propanediols, butane-diols, propanol or mixture thereof.

In another preferred embodiment, 1,2-propanediol is obtained as essential/preferred product from said process.

In a preferred embodiment, the process comprises reacting 1-10 wt. % of sugar with said catalyst in a ratio of 1:0.2 to 1:1, wherein the catalyst is a bimetallic (M1M2) supported onto a support at a temperature in the range of 150° C. to 220° C., rpm in the range of 700 to 1000 rpm, and pressure in the range of 20-70 bars of hydrogen, to obtain value added products; wherein 100% conversion of sugar into value added products is attained; and wherein the M1 is in the range of 3-12% and M2 is in range of 5-30%.

In another preferred embodiment, the process comprises reacting 1-10 wt. % of sucrose with said catalyst in a ratio of 1:0.2 to 1:1, wherein the process comprises of reacting cellulose with catalysts selected from Ni—Mo/Al₂O₃ in the range of Ni varying from 3-12%, Mo varying from 5-30% at a temperature in the range of 150° C. to 220° C., rpm in the range of 700 to 1000 rpm, and pressure in the range of 20-50 bars of hydrogen, to obtain 100% conversion of sucrose.

In another preferred embodiment, the present invention provides a cost-effective, environmental friendly, stable and reusable catalyst for the efficient conversion of sugar(s) into value added product(s), wherein said catalyst is M1-M2/Al₂O₃ with the ratios of M1 varying from 3-12%, and M2 varying from 10-25%.

In another preferred embodiment, the present invention provides a cost-effective, environmental friendly, stable and reusable catalyst for the efficient conversion of sucrose to 1,2-PG, wherein the above said catalyst is Ni—Mo/Al₂O₃ with the ratios of Ni varying from 3-12%, Mo varying from 10-25%.

In an embodiment, the present invention provides a wet impregnation process for the preparation of the catalyst, wherein the process for the preparation of the above catalyst comprises of adding precursor metal solutions in a suitable solvent simultaneously on a support under stirring at a temperature in the range of 60-85° C. for a period of time in the range of 6-8 hr; drying the obtained mixture in an oven at a temperature in the range of 80-120° C. for a period of time in the range of 8-12 hr; followed by grinding and calcining at a temperature in the range of 500-600° C. for 4-6 hr and reducing under hydrogen at a temperature in the range of 350-450° C. for a 4-6 hr to obtain the catalyst.

In another embodiment, the value added products formation as per above process is start as soon as the substrate (sugar or polysaccharide) and catalyst is added with aforesaid reaction conditions (refer, table 6).

Specifically, the reaction time period required for value added products formation is from 0 minutes or 0.1 minutes to 6.5 hrs. More specifically, the reaction time period required for value added products formation is 0, 5, 10, 15, 20, 25, 30, 35 to upto 130 minutes.

In another embodiment, the process provides high EG:PG products yield ratio of about 1:110.

In another embodiment, the catalyst is found reusable for consecutive runs without any change in structural properties of metals and/or support, and the leaching of metals.

In another embodiment, the value added products as obtained herein is optionally purified by literature methods known to a person skilled in the art e.g. washing, drying, evaporation, concentration methods, filtration, chromatographic techniques (conventional or advanced), HPLC, and so on.

In another embodiment, the yield of single value added product is in range of 1 to 79%, and mixture of value added products is in range of 70 to 95% (refer tables 1-3).

In another embodiment, the conversion rate of substrate sugar or polysaccharide into said value added products is of about 100%.

In another embodiment, the value added products obtained by the process provides single isomeric compound (ee) or mixture of isomeric forms/compounds/products from a single reaction.

In another embodiment, the process is done in a batch mode.

Non-noble metals loaded on the support in a catalyst are selected from Ni, Mo, Co, Cu, Fe, W, Sn, Cr, Ce and such like, alone or in combinations thereof.

In a preferred embodiment the non-noble metals are used alone or as a bi-metal combination. The ratio of substrate to catalyst ranges from 1:0.2 to 1:1.

Suitable solvent to dissolve the precursor are selected from polar protic solvent. Polar protic solvent are selected from water, methanol and ethanol. In a particularly preferred embodiment, water is the solvent.

In a preferred embodiment, the process comprises of reacting sugar with catalysts in the ratio of 1:0.2 to 1:1 in a Parr reactor with a solvent, wherein the catalyst is selected from Ni—Mo/Al₂O₃ in the metal ratio of Ni varying from 3-12%, Mo varying from 10-30% at a temperature in the range of 150° C. to 230° C., rpm in the range of 700 to 1000 rpm, and pressure in the range of 20 to 70 bars of Hydrogen, to obtain 100% conversion of sugar to glycols.

The glycols are selected from 1,2-PG, ethylene glycol, n-propane diol, butanediols, lactic acid, propanol and such like.

In an aspect, the process of synthesis of the bi-metallic non-noble catalyst is disclosed. The process for synthesis of the said bimetallic catalyst comprising Ni and Mo is disclosed herein. Accordingly, bimetallic catalysts of Ni and Mo were fabricated by loading nickel and molybdenum transition metals onto the commercially purchased alumina using the conventional incipient wetness impregnation method. Required amount of nickel and molybdenum precursor solution was prepared. The solutions were added on to the alumina support under stirring at a temperature in the range of 60-85° C. The mixture is sonicated 3 to 4 times for 15 minutes to ensure the dispersion of the metal on the catalyst support with a continued stirring for 6-8 h, drying the obtained mixture in an oven at a temperature in the range of 120° C. for a period of time in the range of 8-12 hr; followed by grinding and calcining at a temperature in the range of 500-600° C. for 4 hr and reducing under hydrogen at a temperature in the range of 350-450° C. for a 2-4 hr to obtain the catalyst.

The reduced sample was named as x % M1-y % M2/Al₂O₃ used for the reaction. The x and y are nominal amounts (weight percentages) of metal present in the catalyst.

Another embodiment of the present invention provides a cost-effective, environmental friendly, stable and reusable catalyst for the efficient conversion of sucrose to 1,2-PDO, wherein the above said catalyst is Ni—Mo/Al₂O₃ with the ratios of Ni varying from 3-12%, and Mo varying from 5-30%.

In another embodiment, the present invention provides a wet impregnation process for the preparation of the catalyst, wherein the process for the preparation of the above catalyst comprises of adding precursor metal solutions in a suitable solvent simultaneously on a support under stirring at a temperature in the range of 60-85° C. for a period of time in the range of 6-8 hr; drying the obtained mixture in an oven at a temperature in the range of 80-120° C. for a period of time in the range of 8-12 hr; followed by grinding and calcining at a temperature in the range of 500-600° C. for 4-6 hr and reducing under hydrogen at a temperature in the range of 350-450° C. for a 4-6 hr to obtain the catalyst.

In another embodiment, the catalyst is found reusable for consecutive runs without any change in structural properties of support or the leaching of metals.

Table 1 summarises the metal combination of the catalyst for the selective production of 1,2-PDO at 220° C., 40 bar H₂ pressure for 4.5 h from 2 wt % of sucrose solution at a catalyst ratio of 1:0.35

TABLE 1

| Metal | | | | | | | | | |
|---|---|---|---|---|---|---|---|---|---|
| Loading | | Product Selectivity (From HPLC in %) | | | | | | | |
| (%) | | 1,2- | 1,3- | | 1,2- | 2- | 1- | Lactic | PDO + |
| Ni | Mo | PDO | PDO | EG | BDO | PrOH | PrOH | Acid | BDO |
| 6 | 20 | 70.13 | 1.04 | 4.05 | 15.28 | 1.04 | 1.70 | 2.19 | 86.45 |
| 10 | 20 | 67.07 | 0.36 | 7.22 | 11.41 | 2.86 | 1.79 | 2.40 | 78.84 |
| 8 | 20 | 72.16 | 1.06 | 4.64 | 11.68 | 2.15 | 1.99 | 3.07 | 84.9 |
| 8 | 15 | 61.16 | Trace | 2.92 | 12.03 | 8.22 | Trace | 7.01 | 73.19 |
| 8 | 25 | 69.41 | 0.84 | 5.46 | 12.64 | 1.43 | 1.34 | 2.30 | 82.89 |
| 8 | 0 | 30.36 | 0.63 | 6.30 | 3.14 | 3.55 | Nd | 20.03 | 34.13 |
| 0 | 20 | 15.72 | 1.20 | 14.02 | 2.05 | 4.19 | ND | 32.18 | 18.97 |
| 0 | 0 | 19.92 | 1.34 | 14.60 | 2.19 | 3.68 | ND | 29.75 | 23.45 |

Table 2 below summarizes the effect of catalyst on different substrates at 220° C., 40 Bar H₂ pressure for 4.5 h for the production of 1,2-PDO

TABLE 2

| | Product Selectivity (From HPLC in %) | | | | | | | |
|---|---|---|---|---|---|---|---|---|
| | 1,2- | 1,3- | | 1,2- | 2- | 1- | Lactic | PDO + |
| Substrate | PDO | PDO | EG | BDO | PrOH | PrOH | Acid | BDO |
| Sucrose | 72.16 | 1.06 | 4.64 | 11.68 | 2.15 | 1.99 | 3.07 | 84.9 |
| Fructose | 78.57 | Trace | 3.23 | 7.76 | 1.28 | 0.90 | 3.77 | 86.33 |
| Cellulose | 20.67 | Trace | 16.18 | 6.75 | NA | 1.42 | NA | 28.1 |
| Glucose | 55.76 | Trace | 10.10 | 18.52 | 1.69 | 3.46 | 2.64 | 74.28 |
| Biomass- | 35.26 | Trace | 6.87 | 11.65 | 3.19 | 2.18 | 6.39 | 46.91 |
| Sugarcane | | | | | | | | |
| Bagasse | | | | | | | | |
| Starch | 38.32 | 5.82 | 3.65 | 14.19 | ND | 1.61 | 3.12 | 58.33 |
| powder- | | | | | | | | |
| soluble | | | | | | | | |

Table 3 below summarizes the effect of the different reaction conditions in the production of 1,2-PDO from sucrose

TABLE 3

| Reaction Conditions | | | | Product Selectivity (%) | | | | | |
|---|---|---|---|---|---|---|---|---|---|
| T | P | Sucrose:Catalyst | Time | 1,2- | 1,3- | | 1,2- | Lactic | PDO + |
| (° C.) | (bar) | Ratio | (h) | PD | PD | EG | BDO | Acid | EG + BDO |
| 220 | 40 | 1:0.35 | 4.5 | 72.16 | 1.06 | 4.64 | 11.68 | 3.07 | 89.54 |
| 200 | 40 | 1:0.35 | 4.5 | 71.55 | Trace | 4.62 | 11.91 | 3.23 | 88.08 |
| 180 | 40 | 1:0.35 | 4.5 | 73.72 | 2.65 | 2.50 | 12.25 | 1.82 | 91.12 |
| 160 | 40 | 1:0.35 | 4.5 | 46.04 | 6.04 | 1.93 | 8.68 | Trace | 62.69 |
| 180 | 50 | 1:0.35 | 4.5 | 70.00 | 3.21 | 5.77 | 10.79 | 2.57 | 89.77 |
| 180 | 30 | 1:0.35 | 4.5 | 67.63 | 3.88 | 4.04 | 11.61 | 3.18 | 87.16 |
| 180 | 40 | 1:0.5 | 4.5 | 70.62 | 2.58 | 4.59 | 13.12 | 2.01 | 90.91 |
| 180 | 40 | 1:0.20 | 4.5 | 53.11 | 5.62 | 3.89 | 10.49 | 6.56 | 73.11 |
| 180 | 40 | 1:0.35 | 2.5 | 70.85 | 4.12 | 2.50 | 12.93 | 2.39 | 90.4 |
| 180 | 40 | 1:0.35 | 6.5 | 71.67 | 1.83 | 3.12 | 13.62 | 2.48 | 90.24 |

The investigation of the reusability of the catalyst on the selectivity of the 1,2-PDO has been done at a reaction condition of 2 wt % Sucrose with substrate to catalyst ration of 1:0.35 at 180° C. for 4.5 h at 4 MPa H₂ pressure and is presented in Table 4.

TABLE 4

| | Product Selectivity (%) | | | | | |
|---|---|---|---|---|---|---|
| # of | 1,2- | 1,3- | | 1,2- | Lactic | PDO + |
| Run | PDO | PDO | EG | BDO | Acid | EG + BDO |
| Run 1 | 73.72 | 2.65 | 2.50 | 12.25 | 1.82 | 91.12 |
| Run 2 | 73.19 | 2.57 | 3.82 | 12.79 | 1.65 | 92.37 |
| Run 3 | 69.99 | 2.95 | 3.98 | 10.80 | 3.41 | 87.72 |
| Run 4 | 61.81 | 1.69 | 4.28 | 11.63 | 6.62 | 79.41 |

In a preferred embodiment, the process results in substantially high yield 1,2-PG, facilitating its separation from other products. This aspect overcomes the drawback of prior art reported processes, wherein a mixture of diols are obtained.

In an aspect of the invention, the process of conversion of sugars and polysaccharides into value added products can be done in a continuous process.

The disclosed process provides a cost-effective, environmental friendly, stable and reusable catalyst for the efficient conversion of sucrose to 1,2-PG. The present invention provides 100% conversion of sucrose into valuable products such as polyols with high selectivity under mild reaction conditions.

Specifically, the inventors of present invention have developed specific catalysts with various amounts of Ni—Mo loaded on γ-Al₂O₃ support for testing the catalytic conversion of sucrose to 1,2-PDO. The phases and the textural properties of the prepared Ni—Mo/g-Al₂O₃ catalysts were analysed by XRD and BET analysis. The morphology, particle size, and metal distribution of the catalysts were analysed by FE-SEM, HR-TEM, and HAADF-STEM techniques. The catalytic conversion of sucrose to 1,2-PDO over Ni—Mo loaded g-Al₂O₃ was studied. The 8% Ni-20% Mo/g-Al₂O₃ catalyst gave the selectivity of 1,2-PDO as high as ˜74% under milder reaction conditions. The presence of the optimum amounts of Lewis and Brönsted acid sites over 8% Ni-20% Mo/g-Al₂O₃ helps in C—C bond cleavage, leading to more 1,2-PDO selectivity with complete conversion. The catalyst was stable after four consecutive runs without any changes in morphology.

### EXAMPLES

General Information: HPLC analysis is carried out in Perkin Elmer series 500 instrument using manual injection and the datacollection is done using RID and TC NAV software. The column using for the analysis is Rezex Organic Acid Column with 0.005M H₂SO₄ in millipore water as mobile phase. The run time of analysis is 40-60 minutes at 0.5 ml min-1 flow rate and column temperature of 60° C.

Source of substrates: Cellulose (Microcrystalline 20 μm, Aldrich Chemistry), Sucrose (Extra pure, LobaChemie), D-(−)-Fructose (>99.0%, Sigma) and D-(+)-Glucose (>99.5%, Sigma), Starch powder (S.d. fine chem. ltd)

Following examples are given by way of illustration and therefore should not be construed to limit the scope of the invention.

### Example 1: General Process for the Preparation of the Catalyst

Metal precursor-1 was dissolved in DI water in a beaker and metal precursor-2 was separately dissolved in DI water in another beaker by stirring. Both the solutions were simultaneously added on to the support at 80° C. under stirring for 6-8 hours. The mixture was sonicated 3-4 of times in between for 15-30 minutes each. A little amount of DI water was added in between to make up with the loss of water, if any. After 8 hours, the mixture was kept for drying in oven at 110° C. for 10-12 hr. The dried catalyst was ground into fine powder and calcined in Muffle furnace at 550° C. with the ramp rate of 2° C. min-1 for 4 hours. The calcined sample was then reduced under hydrogen atmosphere in tubular furnace at 400° C. with ramp rate of 5° C. min⁻¹ for 4 hours. The reduced sample was used for the reaction.

### Example 2: General Method for Conversion of Sucrose as Substrate into 1,2-Propanediol or Mixture of Value Added Products

The activity of the prepared catalyst on cellulose conversion was studied on 50 ml Parr SS Batch Reactor (5500 series with 4848 Controller). The catalyst and substrate were taken at the ratio of 0.35:1 in Parr reactor with sufficient amount of water to make it 4 wt % of sucrose solution. The system was first purged with Nitrogen and then with Hydrogen gas. After purging, the reactor was pressurized to 40 bar (30-50 bar based on reaction requirements) pressure at 20° C. and heated upto 180° C. (160-180° C. based on reaction requirements). The reaction was maintained with constant stirring (700-1000 rpm based on reaction requirements) for a period of 2.5-6.5 hr.

After the reaction was completed, the stirring and heating was switched off and the system was allowed to cool on its own. The product mixture was then filtered, the catalyst was recovered and the product solution was analyzed using HPLC.

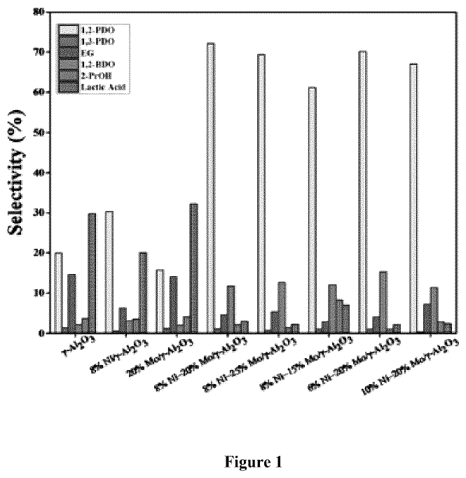

FIG. 1 confirms the production of mixture of value added products containing C2 and/or C3 diols, monools or acids with higher abundance or preferred yield of 1,2-propanediol.

### Example 3: General Method for Production of Value Added Products Using Different Substrates

The substrate used instead of sucrose is cellulose (Microcrystalline 20 mm, Aldrich Chemistry), D-(−) fructose (>99.0%, Sigma), D-(+)-glucose (>99.5%, Sigma) and sugarcane bagasse, for production of value added products. In a typical experiment, the calculated amounts of the substrate (any one from said substrates) and the catalyst (of example 1) were added to the reactor vessel with the required amounts of DI water according to the substrate: catalyst ratio. The system was first purged with nitrogen thrice and then with hydrogen gas. The reactor with the reaction mixture was then pressurized to 30 to 50 bar H2 pressure at room temperature (at 25° C.). The reaction was carried out at a temperature ranging from 160° C. to 220° C. for a period of 2.5 h to 6.5 h. After the reaction completed, the liquid products were separated from the solid products by filtration. The products in the solution were analyzed using high-performance liquid chromatography (HPLC, PerkinElmer Series 200) instrument with a refractive index (RI) detector. The separation of the product mixture was achieved using a REZEX ROA (H+ organic acid) column, and 0.005 M of H2SO4 was used as the mobile phase with a flow rate of 0.5 mL-min-1.

Table 5 provides % product selectivity of all substrates into said value added products (yield in %), as below:

TABLE 5
8% Ni-20% Mo/γ-Al2O3 catalyst over different substrates
at 220° C., 40 bar H2 for 4.5 h at 1000 rpm^{a}

| | Product selectivity (%) | | | | | | | |
|---|---|---|---|---|---|---|---|---|
| | 1,2- | 1,3- | | 1,2- | 2- | 1- | Lactic | PDO + |
| Substrate | PDO | PDO | EG | BDO | PrOH | PrOH | acid | BDO |
| Sucrose | 72.2 | 1.1 | 4.6 | 11.7 | 2.2 | 2.0 | 3.1 | 85.0 |
| Fructose | 78.6 | Trace | 3.2 | 7.8 | 1.3 | Trace | 3.8 | 86.4 |
| Cellulose | 20.7 | Trace | 16.2 | 6.8 | NA | 1.4 | NA | 27.5 |
| Glucose | 55.8 | Trace | 10.1 | 18.5 | 1.7 | 3.5 | 2.6 | 74.3 |
| Real | 35.3 | Trace | 6.9 | 11.6 | 3.2 | 2.2 | 6.4 | 46.9 |
| sugarcane | | | | | | | | |
| biomass | | | | | | | | |

^{a}All the reactions showed 100% conversion. The products include 1,2-propanediol (1,2-PDO), 1,3-propanediol (1,3-PDO), ethylene glycol (EG), 1,2-butanediol (1,2-BDO), 2-propanol (2-PrOH), 1-propanol (1-PrOH) and lactic acid.

It was observed that the fructose (monosaccharide) gave the highest amount of 1,2-PDO. Unlike fructose, sucrose being a disaccharide, the selective production of the C3 glycol might be more difficult. The glucose, cellulose, and the sugarcane bagasse have less selectivity to 1,2-PDO. Hence, the further optimisation of the reaction conditions of the catalyst was carried out using sucrose as the substrate.

The inventors have further carried out studies which shown that the catalyst is active even from the very start of the reaction and is as provided in the Table 6:

TABLE 6
Reaction condition: 2 wt % Sucrose, 180° C., 4 MPa H₂ pressure

| | Time of | Sucrose | EG | PG | EG:PG |
|---|---|---|---|---|---|
| Sl No | study | conversion | selectivity % | Selectivity % | ratio |
| 1 | 0 minutes | 64.7 | 0.4 | 44.2 | 1:110 |
| 2 | 15 minutes | 78.8 | 0.8 | 52.8 | 1:66 |
| 3 | 30 minutes | 85 | 1.1 | 54.9 | 1:50 |
| 4 | 45 minutes | 91.1 | 1.4 | 57.9 | 1:41 |
| 5 | 60 minutes | 95.1 | 2.0 | 59.2 | 1:30 |
| 6 | 75 minute | 97.6 | 1.7 | 64.1 | 1:38 |
| 8 | 105 minute | 98.8 | 2.4 | 62.7 | 1:26 |

From the above table 6, it is clear that the process is highly active from the 0th minute or milliseconds of reaction resulting in the high EG:PG ratio of 1:110 which is ever reported. As mentioned, 74% yield is the highest 1,2-PDO selectivity ever reported from a carbohydrate source (specifically sucrose).

### Characterization:

After the reaction, the liquid products were separated from the solid products by filtration. The products in the solution were analyzed using high-performance liquid chromatography (HPLC, PerkinElmer Series 200) instrument with a refractive index (RI) detector. The separation of the product mixture was achieved using a REZEX ROA (H⁺ organic acid) column. 0.005 M of H₂SO₄ was used as the mobile phase with a flow rate of 0.5 mL min-1. A typical analysis run lasted for 40 min. The column temperature was maintained at 60° C. throughout the analysis.

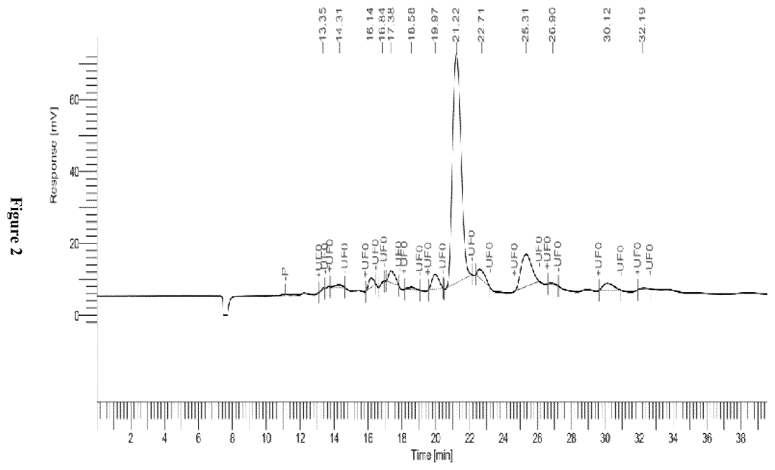

As per FIG. 2 of HPLC graph, the peak at 21.2 confirms the 1,2-PDO, the peak at 25.31 confirms 1,2-BDO, the peak at 19.9 confirms the EG, the peak at 16.1 confirms the lactic acid, the peak at 13.3 confirms the sorbitol/manitol, the peak at 17.3 confirms the glycerol, and the peak at 22.7 confirms 1,3-PDO.

The notation (−P) in FIG. 2 indicates the Peak Disabled, which is required for the manual integration as the instrument picks up Noise also as the peak.

The notations (+UFO) and (−UFO) in FIG. 2 indicate User forced peak start and User forced peak stop.

### Advantages of the Invention

The present process is simple, energy efficient and economical.
The synthesis of catalyst is by a very simple wet impregnation process which is inexpensive and environmentally friendly.
100% conversion of cellulose or other substrates into high yield of valuable products at low temperature and optimum pressure, is obtained.
Catalyst is recyclable.
The catalyst support is less expensive.
The selectivity of propylene glycol is about 50 to 70 times than provided in aforesaid report Arun Arunima Kirali et al. Also, the charring resulted in the reaction of this article makes it unfit for the commercialisation, while process of present invention did not find any charring.
Provides wide variety of substrates for production of value added products such as 1,2-propanediol, ethylene glycol, etc.
No use of any noble (costly) metals in bimetallic catalyst system.
The aforesaid process is efficient in terms of catalyst cost, milder reaction conditions and energy efficient separation.
The present invention overcomes major challenge in considering glycol production is the separation of glycols specially ethylene glycol (EG) and propylene glycol (PG) because of their close boiling point and formation of azeotropic mixture. In present invention, inventors have seen a remarkable ratio of EG:PG of about 1:30 which is far ahead of the reported ratios in any of the literatures provided.
Ni—Mo/Alumina is a cheap catalyst in terms of economic aspects. The high activity of the catalyst and its stability and reusability makes the process more superior and better.
The other products like 1,2-butanediol, lactic acid and other glycol produced along with the reaction also have high market value.
No kind of organic solvents or acids are used in this reaction other than water as the reaction medium. This makes the process environmental friendly, with minimal chemical component utilisation.
The amount of catalyst used is as low as 200 to 350 mg for 1000 g of substrate.

1. A one pot-one step process for preparation of products from sugar(s) or polysaccharide(s), the process comprising:
mixing a reaction mixture comprising said sugar(s) or polysaccharide(s) and a catalyst containing non-noble metal(s) supported onto a support, in a solvent under H2 gas, at pressure in the range of 20,000,000 to 70,000,000 Pascal (20 to 70 bar) at temperature of 20 to 30° C., followed by heating at a temperature in the range of 150 to 220° C. and stirring at a speed in the range of 7000 to 1000 rpm for a time period in the range of 1 minute to 6.5 hrs to obtain the products;
wherein the products are selected from polyol/diol, monool (monohydroxy alcohol), acid, or mixtures thereof, and
wherein conversion rate of said sugar(s) or polysaccharide(s) into the products is 100%.

2. The process as claimed in claim 1, wherein the products is/are selected from 1,2-propanediol, 1,3-propanediol, ethylene glycol, 1,2-butanediol, 2-propanol, 1-propanol, lactic acid, or mixtures thereof.

3. The process as claimed in claim 1, wherein yield of a single product is in a range of 1 to 79% and yield of a mixture of the products is in a range of 70 to 95%.

4. The process as claimed in claim 1, wherein the non-noble metal(s) loaded onto the support in the said catalyst is selected from nickel (Ni), molybdenum (Mo), cobalt (Co), copper (Cu), iron (Fe), tungsten (W), tin (Sn), chromium (Cr), cerium (Ce), or combinations thereof.

5. The process as claimed in claim 1, wherein the catalyst is a bimetallic system supported onto a support, comprising M1M2 metals supported onto said support; and wherein M1 is selected from nickel (Ni), cobalt (Co), copper (Cu), and Iron (Fe); and M2 is selected from molybdenum (Mo), tungsten (W), Lanthanum (La), tin (Sn), Cerium (Ce), and chromium (Cr).

6. The process as claimed in claim 5, wherein weight % of M1 in said catalyst is in a range of 3-12 wt. %; the weight % of M2 in said catalyst is in a range of 5-30 wt. %; and the weight % of support in said catalyst is in a range of 58 to 92 wt. %.

7. The process as claimed in claim 1, wherein the support is selected from alumina (γ-Al₂O₃), zeolite and mesoporous carbon.

8. The process as claimed in claim 1, wherein the solvent used to dissolve the said sugar(s) or polysaccharide(s) with a catalyst is selected from a polar protic solvent; and wherein the polar protic solvent is selected from water, methanol, ethanol, or mixtures thereof.

9. The process as claimed in claim 1, wherein a ratio of said sugar(s) or polysaccharide(s) and said catalyst is in a range of 1:0.2 to 1:1.

10. The process as claimed in claim 1, wherein the sugar is selected from monosaccharides or disaccharides; wherein the sugar is selected from pentose(s) or hexose(s); and wherein the polysaccharide is selected from cellulose, biomass, or starch.
